(19) United States
(12) Patent Application Publication
(10) Pub. No.: US 2022/0339078 A1
(43) Pub. Date: Oct. 27, 2022

(54) Indirect Barrier Intervention Method and Apparatus

(71) Applicant: Aubrey Russell Hopkins, Tallahassee, FL (US)

(72) Inventor: Aubrey Russell Hopkins, Tallahassee, FL (US)

(21) Appl. No.: 17/860,858

(22) Filed: Jul. 8, 2022

Related U.S. Application Data
(62) Division of application No. 16/537,061, filed on Aug. 9, 2019, now abandoned.
(60) Provisional application No. 62/862,921, filed on Jun. 18, 2019.

(51) Int. Cl. A61K 6/69; A61K 9/00; A61K 8/21; A61Q 11/00

(52) U.S. Cl. CPC A61K 6/69; A61K 9/0063; A61K 8/21; A61Q 11/00; A61L 2300/404; A61L 26/0066

(57) ABSTRACT

A dental device that provides for the entirely non-surgical management of early, cavitated dental lesions consisting of a ceramic wafer sized to cover the dental lesion and resin bonded to the tooth effectively encapsulating the dental lesion. The ceramic wafer may incorporate antimicrobial preparations, radiopaque markers, and/or silver diamine fluoride.

### CROSS REFERENCE TO RELATED APPLICATIONS

This application is a divisional under 35 USC 119 (e) from U.S. patent application Ser. No. 16/537,061 filed Aug. 9, 2019, which claims priority of U.S. Provisional Patent Application 62/862,921 filed Jun. 18, 2019.

### STATEMENT REGARDING FEDERALLY SPONSORED RESEARCH OR DEVELOPMENT

Not Applicable

### MICROFICHE APPENDIX

Not Applicable

### BACKGROUND OF THE INVENTION

### 1. Field of the Invention

This invention relates to the field of dental devices for the treatment of dental lesions. More specifically, the present invention is a dental device that provides for the entirely non-surgical management of early, cavitated interproximal (the interproximal or approximating surfaces are those between one tooth and its neighbor) dental lesions.

### 2. State of the Art

In industrialized countries most of the adult population has one or more carious lesions that have been treated with fillings. Each carious lesion, which can ultimately lead to a surface cavitation (cavity), begins with a demineralization of the hard tooth substance. In the early stage, the caries lesion is referred to as the incipient. enamel caries, Upon clinical investigation the tooth surface appears to remains intact. If lesion progression continues, clinical surface changes occur and the affected enamel will demonstrate a clinically (visibly) evident “white lesion”. A white lesion is a cardinal sign of demineralization. It provides evidence of the affect as the refraction of light upon a demineralized surface is altered. However, the degree of surface demineralization present of interproximal surfaces cannot be seen. A series of radiographs combined with an understanding of a patient's risk of additional disease serve to guide a practitioner's recommendations for treatment. Not arrested interproximal lesions may progress without visible signs of erosion but the demineralized area below the surface becomes more and more porous.

At present, active and progressing interproximal dental lesions that evidence demonstrate continuing demineralization progression over time after currently available standard of care remineralization strategies are exhausted (i.e., there is irreversible surface damage) are managed surgically by the cutting away of the lesion, all or in part, and in almost all cases concurrent with the surgical removal of healthy and unaffected tooth structure. The resulting “preparation” is then filled with one of the many and various dental products (restorative materials) to compensate for the loss of tooth structure. The planned “filling”, it is hoped, will artificially restore the preponderance of lost tissue while respecting the anatomy of the original tooth and the vitality of the dental pulp. This simple operation yields results that are frequently less than optimal. The selected restorative material may be poorly placed or delivered, poorly condensed, compacted, contoured or manipulated. It may have voids at critical material-tooth interfaces that are not discernable clinically or radiographically. The material's physical qualities may be compromised by operator error via mishandling or unknowingly violating a manufacturer's criteria for successful use.

Many restorative resin composite and resin bonding systems are extremely delicate and unforgiving and many seek to overcome the challenges and complications of managing a resin interface with the tooth's hard tissues dentine. These complications include moisture contamination and the generation of inflammatory radicals that may compromise the physical characteristics of a restoration and may elicit an unwanted pulpal response. The majority of class II (interproximal) restorations place physical, mechanical stress on the tooth, and provide surface irregularities that at micro levels provide regions for bacterial colonization. These restorations often physically fail over an unacceptably short period of serviceability. An indirect barrier intervention device avoids all of the aforementioned complications. It is entirely bonded to enamel and has no working interface with dentin. Therefore, methods of treating caries demonstrating initial surface cavitation at an early state, in particular approximal, incipient carious lesions, are highly desirable to avoid the subsequent need for invasive procedures.

Thus, there is a need for an immediate, non-invasive, non-surgical dental intervention that provides a safe, effective, long-term treatment for the management of cavitated dental lesions.

### 3. Summary of the Invention

The present invention is a small, thin, transparent (or semi-opaque) crystalline wafer, lozenge, or patch that creates an entirely external dental bandage. This dental bandage is tenaciously bonded to dental enamel and situated for the purpose and effect of denying the introduction of substrate (dietary sugars) to the lesion body (the surface cavity and the biologic damage behind it). This dental bandage may be used for the effective and long term, non-surgical management of early approximal smooth surface cavitated carious lesions. The dental bandage is referred to herein as “DB” and its method of use is referred to herein as an Indirect Barrier Intervention (“IBI”).

In one embodiment, the DB is a spherical or rectangular chip of a material such as sapphire or corundum. The DB will present an impervious/non-soluble external surface and a mechanically and/or chemically retentive working internal surface that is meant to be intimate with the tooth surface to be protected.

The working (internal or interfacing) DB surface is treated as required to achieve maximum bond strengths with enamel when bonded. The external DB surface is polished smooth. After bonding the DB to enamel any extraneous bonding material is to be removed by a process such as air abrasion with glycerin powder or any other suitable procedure known in the art.

This summary provides, in simplified forms, concepts that are more fully described and detailed below. This summary is not intended to identify key features or essential features of the claimed subject matter, nor is this summary intended to be used as an aid in determining the scope of the claimed subject matter. Additional features and advantages of the invention will be set forth in the following description, or may be learned by the practice of the invention. The features and advantages of the invention may be realized and obtained by means of the appended claims. These and other features of the present invention will become more fully apparent from the following description and appended claims, or may be learned by the practice of the invention as set described in this application.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 is a view of a tooth with a caries lesion.

FIG. 2 is a view of a tooth with a caries lesion and a DB deployed over the lesion.

FIG. 3 is a side view of the DB applied to a tooth.

FIG. 4 is a view of the tool for transporting a DB.

### DETAILED DESCRIPTION OF THE INVENTION

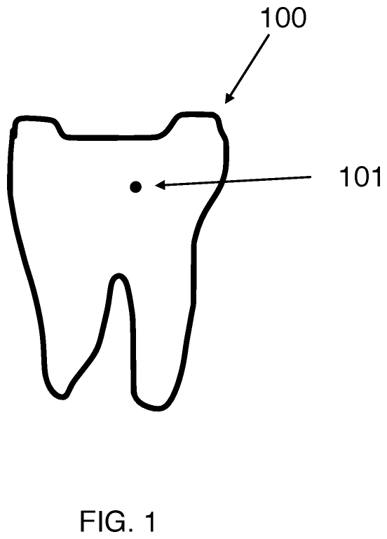

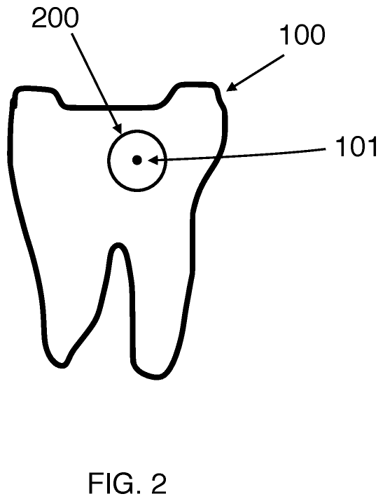

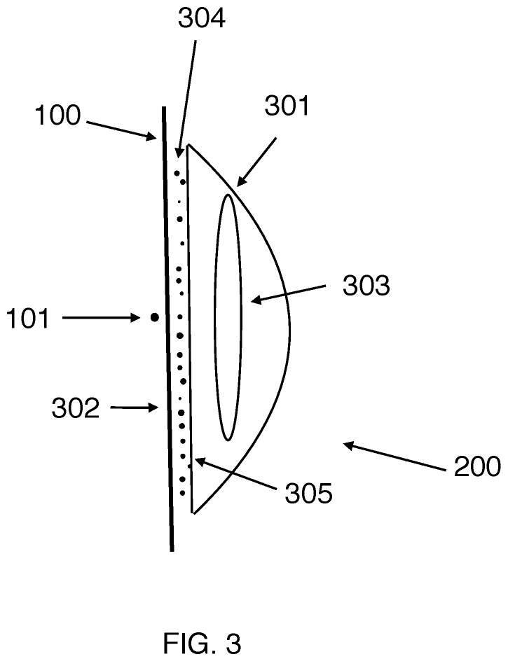

As illustrated in FIG. 1, a tooth 100 is shown with a caries lesion (or cavity) 101. As shown in FIG. 2, tooth 100 is shown with a dental bandage (“DB”) 200 applied over cavity 101. DB 200 is preferably a small, thin, transparent (or semi-opaque) crystalline wafer, lozenge, or patch. Preferably, the DB may be comprised of a compressible, glass ionomer wafer with a maximum thickness of less than 50 mu and a minimum edge thickness of approximately 5 mu. Alternatively, the DB may be formed from any suitable crystalline material such as sapphire or aluminum oxide. As shown in FIG. 3, DB 200 includes an exterior surface 301 that is impermeable, non-soluble, biocompatible, hard, smooth, and highly polished, and is produced and shaped in the form if a low profile spherical cap or lens. Optionally, DB 200 may have a square, rectangular, triangular, or other geometric shape. DB 200 further includes an interior surface 305 (in contact with tooth 100 via bonding material 304) that is treated or worked to enhance bonding to tooth 100 enamel. This may involve one or more processes such as the inclusion of alumina or mono/polycrystallines, or mechanical treatments resulting in a silanated, etched, or abraded surface to enhance mechanical and chemical bonding between the DB 200 and tooth 100 enamel. DB 200 may further include a compressible mid-section 303 that allows for flexibility in the placement and bonding of DB 200.

Optionally, DB 200 may include a radiopaque marker to evidence the presence and location of the DB 200. DB 200 may further incorporate therapeutic materials such as glass ionomer or silver diamine fluoride that directly interface with the cavitated tooth enamel surface prior to placement of DB 200.

DB 200 preferably has a maximum height of contour of less than 500 mu and is sized (height and width dimensions) to be just larger than the physical, clinical presentation radiographic image of the clinically disclosed surface cavitation 101. DB 200 encapsulates, strengthens, and protects tooth 100 once applied.

As illustrated in FIG. 1, a tooth is shown with an early carious lesion with surface cavitation. The tooth surface is prepared (preferably by cleaning and polishing followed by acid etching) to receive the DB. Bonding material 304 affixed to the interior side of the DB is then placed in contact with tooth enamel and cured by photo initiation followed by a chemical cure. Preferably, the DB is resin bonded to the tooth enamel.

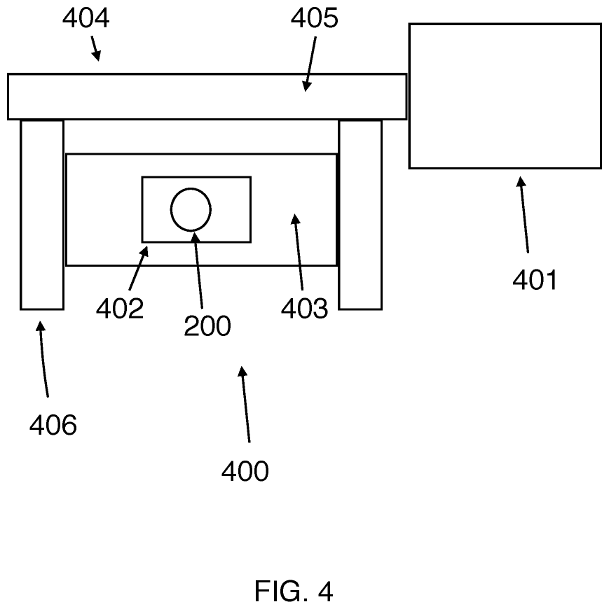

As seen in FIG. 4, the carrier 400 for a DB 200 is shown. Preferably, the carrier consists of a handle 401 that is removably attachable to a frame structure 404. The frame structure 404 includes a horizontal beam 405 and two vertical guides 406 extending downward from the horizontal beam 405. Connected between the vertical guides 406 is carrier film 403, composed of a clear plastic such as mylar, that is sufficiently strong and resilient for its intended purpose. Any suitable clear film may be used in this application. In operation, a DB 200 is weakly bonded to DB patch 402 and DB patch 402 is weakly bonded to carrier film 403. The carrier 400 is then inserted into a patient's mouth to position the DB 200 over the effected tooth.

It will be readily seen by one of ordinary skill in the art that the present invention fulfills all the objects set forth above. After reading the foregoing specification, one of ordinary skill will be able to effect various changes, substation of equivalents and various other aspects of the invention as broadly disclosed herein. It is, therefore, intended that the protection granted hereon be limited only by the definition contained in the appended claims and equivalents thereof.

What is claimed is:

1. A method for treating an initial surface cavitation on a tooth comprising:
detecting the initial surface cavitation on the tooth;
providing a preformed dental bandage having an outer surface and an inner surface, said preformed dental bandage configured to cover the initial surface cavitation and reduce or prevent introduction of dietary sugars to the initial surface cavitation; and
adhering the inner surface of the preformed dental bandage to enamel on a hard tooth surface of the tooth to cover the initial surface cavitation, thereby reducing or preventing introduction of dietary sugars to the initial surface cavitation.

2. The method for treating a dental caries lesion according to claim 1, wherein the dental bandage is not adhered to dentin of the tooth.

3. The method for treating a dental caries lesion according to claim 1, wherein the dental bandage comprises aluminum oxide.

4. The method for treating a dental caries lesion according to claim 1, wherein the dental bandage is configured to cover a minority portion of the hard tooth surface.

5. The method for treating a dental caries lesion according to claim 1, wherein the dental bandage incorporates an antimicrobial agent.

6. The method for treating a dental caries lesion according to claim 1, wherein the dental bandage is transparent or semi-opaque.

7. The method for treating a dental caries lesion according to claim 1, wherein the dental bandage is adhered the tooth surface via resin bonding.

8. The method for treating a dental caries lesion according to claim 1, further comprising etching the enamel on the hard surface of the tooth prior to the adhering.

9. The method for treating a dental caries lesion according to claim 1, wherein the dental bandage includes a radiopaque marker.

10. The method for treating a dental caries lesion according to claim 1, wherein inner surface of the dental bandage incorporates an effective amount of silver diamine fluoride.

11. A method for treating a dental caries lesion on a tooth comprising:
providing a preformed dental bandage having an outer surface and an inner surface, said preformed dental bandage configured to cover the dental caries lesion and reduce or prevent introduction of dietary sugars to the dental caries lesion, the preformed dental bandage having a maximum height of contour of less than 500 μm; and
adhering the inner surface of the preformed dental bandage to enamel on a hard tooth surface of the tooth to cover the dental caries lesion, thereby reducing or preventing introduction of dietary sugars to the dental caries lesion.

12. The method for treating a dental caries lesion according to claim 11, wherein the dental bandage is not adhered to dentin of the tooth.

13. The method for treating a dental caries lesion according to claim 11, wherein the dental bandage comprises aluminum oxide.

14. The method for treating a dental caries lesion according to claim 11, wherein the dental bandage is configured to cover a minority portion of the hard tooth surface.

15. The method for treating a dental caries lesion according to claim 11, wherein the dental bandage incorporates an antimicrobial agent.

16. The method for treating a dental caries lesion according to claim 11, wherein the dental bandage is transparent or semi-opaque.

17. The method for treating a dental caries lesion according to claim 11, wherein the dental bandage is adhered the tooth surface via resin bonding.

18. The method for treating a dental caries lesion according to claim 11, further comprising etching the enamel on the hard surface of the tooth prior to the adhering.

19. The method for treating a dental caries lesion according to claim 11, wherein the dental bandage includes a radiopaque marker.

20. The method for treating a dental caries lesion according to claim 11, wherein inner surface of the dental bandage incorporates an effective amount of silver diamine fluoride.
